(19) United States
(12) Patent Application Publication
(10) Pub. No.: US 2022/0079793 A1
(43) Pub. Date: Mar. 17, 2022

(54) KNEE ORTHOSIS

(71) Applicant: Ottobock SE & Co. KGAA, Duderstadt (DE)

(72) Inventors: Klaus Lidolt, Duderstadt (DE); Tim Kruchem, Göttingen (DE)

(73) Assignee: Ottobock SE & Co. KGAA, Duderstadt (DE)

(21) Appl. No.: 17/415,596

(22) PCT Filed: Dec. 12, 2019
(86) PCT No.: PCT/EP2019/084792

§ 371 Date: Jun. 17, 2021

(30) Foreign Application Priority Data
Dec. 19, 2018 (DE) 10 2018 132 957.1

(51) Int. Cl. A61F 5/01

(52) U.S. Cl. CPC A61F 5/0125

(57) ABSTRACT

The invention relates to a knee orthosis which comprises a ventral and a dorsal section, an upper leg contact element, a lower leg contact element and a knee element, at least one bracing element and at least one inelastic strap, characterized in that the upper leg contact element and the lower leg contact element are connected to the knee element by the at least one bracing element in the ventral region and by the at least one inelastic strap in the dorsal region, or vice-versa.

The invention relates to a knee orthosis which comprises a ventral and a dorsal section, an upper leg contact element, a lower leg contact element and a knee element as well as at least one bracing element and at least one inelastic strap. Such knee orthoses have been known as osteoarthritic orthoses within the scope of the prior art for many years. They can also be used to treat knee deformities, especially genu varum and genu valgum. These deformities differ in the knee frontal angle of the leg, that is, the angle between the longitudinal direction of the upper leg and the longitudinal direction of the lower leg, which is in the frontal plane. In colloquial terms, they are referred to as “X” and “0” legs. An orthosis for these applications often also has a knee frontal angle between the upper leg direction and the lower leg direction. This knee frontal angle then also lies in the frontal plane.

Corresponding knee orthoses are described in U.S. Pat. No. 6,981,957 B2 for example. They should be able to be used for genu varum deformities as well as for genu valgum deformities. Such knee orthoses often feature upper leg rails and lower leg rails made from a rigid material, such as a metal. These rails are often connected in a joint that is designed as a swivel joint whose swivel axis in the mounted state of the knee orthosis coincides or at least almost coincides with the compensating swivel axis of the knee joint. Although this rigid design allows a relatively large force to be applied to the different regions of the leg, the orthosis is bulky, uncomfortable and thus only has a low degree of acceptance.

U.S. Pat. No. 7,662,122 B2, on the other hand, discloses a knee orthosis that also has a three-point effect to treat the aforementioned deformities. It features an upper leg contact element and a lower leg contact element, which are mounted respectively on the upper leg and the lower leg in order to apply a compressive force to them. This upper leg contact element and the lower leg contact element are arranged either medially or laterally, but always on the same side. On the opposite side, the knee element is arranged at the level of the orthosis wearer's knee joint in order to apply a compressive force in the opposite direction as the third adjoining element. Depending on the positioning of the individual elements, such an orthosis can be used to treat both genu varum deformities and genu valgum deformities.

To be able to adjust this force, the knee orthosis has at least one bracing element and at least one inelastic strap, by way of which the various elements are connected to each other. In the orthosis known from U.S. Pat. No. 7,662,122 B2, the bracing element is in the form of a tension wire, the length of which can be changed with a twist lock. The element itself has two loops, one of which connects the knee element to the lower leg contact element and the other of which connects the knee element to the upper leg contact element. One section of each of these loops extends in the ventral part of the knee orthosis and another section in the dorsal part. As a result, a uniform pressure can be applied to the leg by the respective elements.

However, it is disadvantageous that in this way the positions of the upper leg contact element and the lower leg contact element on the respective upper leg or lower leg cannot be adjusted. A shortening of the at least one bracing element always results in a symmetrical force distribution, so that although the force applied to the leg by the respective pressure pad can be increased or decreased, the force application point always remains the same. In addition, due to the fact that the respective bracing elements always have to be guided around the wearer's leg, the knee orthosis is structurally complex and therefore costly.

The invention aims to eliminate or at least mitigate these disadvantages of the prior art. The invention solves the problem by way of a knee orthosis according to the preamble of claim 1, which is characterized in that the upper leg contact element and the lower leg contact element are connected to the knee element by the at least one bracing element in the ventral region and by the at least one inelastic strap in the dorsal region or vice versa.

Consequently, in knee orthoses according to the invention, the contact element and the lower leg contact element are also connected to the knee element by the at least one bracing element. However, unlike with the prior art, this connection is either in the ventral region or the dorsal region. This has various advantages. On the one hand, it is particularly easy to establish the connection between the bracing element and the element or contact element. Loop-like bracing elements, as known from the prior art, are not necessary. Rather, one end of the at least one bracing element can be arranged on the upper leg contact element and another end on the lower leg contact element, which can be done, for example, by simple knotting, screwing on, welding on or other connection. It is not necessary to arrange extensive and long loops in the material of an orthosis base body, where applicable. In addition, no bracing element is provided on the respective opposite side where the upper leg contact element and the lower leg contact element are connected to the knee element by the inelastic strap. This may be advantageous in the ventral region if the ventral region, i.e. the front side of the leg, is subjected to a load, for example, when sitting, haptic impressions are to be taken or other activities are to be carried out for which the positioning of a bracing element, in particular in the form of a tension wire, is uncomfortable, possibly even painful and therefore little accepted. Alternatively, this may also be the case in the dorsal region, which is especially advantageous if the knee orthosis is used by a wearer who uses it for long periods of sitting. Sitting for too long on, for example, a bracing element designed as a tension wire is uncomfortable, can lead to painful skin irritations, therefore causing a low degree of acceptance of the knee orthosis.

Moreover, the unilateral arrangement of the bracing element on either the dorsal side of the ventral side means that the tension can be changed on only one side. If, for example, the length of the bracing element is changed, for instance shortened, not only the force applied by the respective element or contact element changes, but it is possible to simultaneously ensure that the respective element itself is displaced to a certain extent along the leg, i.e. the upper or lower leg, of the wearer, so that the point of application of the transmitted force also changes. If this is not desired, this can be compensated for by also designing the at least one inelastic strap to be variable in length.

The upper leg contact element is preferably an upper leg pressure pad. The lower leg contact element is preferably a lower leg pressure pad. The knee element is preferably a knee pressure pad.

The upper leg contact element and the lower leg contact element are preferably connected to each other by a connecting element, such as a rod, by way of which compressive forces can be transmitted. The connecting element extends either on the medial or lateral side, i.e. on the side where the upper leg contact element and the lower leg contact element are arranged. This prevents the force applied by the bracing element from causing a displacement of the upper leg contact element and/or the lower leg contact element towards the wearer's knee.

The connecting element is preferably designed to be flexible in a direction perpendicular to the direction of extension running from the lower leg contact element to the upper leg contact element and/or has a corresponding joint. This ensures that the connecting element, which extends beyond the knee joint of the wearer, can follow a movement of the knee and does not impede it or only does so very slightly.

Preferably, the knee orthosis has precisely one bracing element which extends from the upper leg pressure pad via the knee pressure pad to the lower leg pressure pad and/or it has an inelastic strap which extends from the upper leg contact element via the knee element to the lower leg contact element. This bracing element and/or the inelastic strap are preferably designed to be variable in length.

In a preferred embodiment, the knee orthosis features an adjustment element by way of which this length of the at least one bracing element can be adjusted, wherein the adjustment element is preferably situated on the knee element or is the knee element.

The adjustment element is, for example, an adjustment element known from the prior art, for example as is known from WO 2014/124054 A1.

Of course, other adjustment elements are conceivable. In particular, in the case where a single bracing element is used, the construction is particularly simple, as the one end of the bracing element can be arranged on the upper leg contact element, the other end on the lower leg contact element, and the adjustment mechanism on the knee element.

In a preferred embodiment, the knee orthosis has at least two bracing elements, of which at least one extends from the upper leg contact element to the knee element and at least one extends from the knee element to the lower leg contact element and/or at least two inelastic straps, of which at least one extends from the upper leg contact element to the knee element and at least one extends from the knee element to the lower leg contact element. In an especially preferred embodiment, the inelastic strap features at least one, but especially preferably two adjustment elements for adjusting the lengths of the inelastic strap, of which especially preferably one adjustment element is located between the upper leg contact element and the knee element and the other adjustment element is located between the knee element and the lower leg contact element.

Each of the bracing elements and/or each of the inelastic straps preferably has an end that is securely mounted on one of the respective connected components, i.e. knee element, upper leg contact element and lower leg contact element. The respective other end of the bracing element and/or the inelastic strap is equipped with an adjustment element, by way of which it can be wound up, for example, in order to shorten the length. These adjustment elements are preferably arranged on the knee element and can be positioned one above the other. In this case, one above the other means, starting from the patient's knee, one above the other in the radial direction, i.e. laterally or medially adjacent.

The at least one bracing element and/or the at least one strap preferably features at least one strap, at least one wire, at least one cable, at least one toothed rail construction and/or cable pull. Here, a toothed rail construction works like a ratchet mechanism, for example. The toothed rail features a plurality of teeth that engage in a ratchet element. The rail can only be moved in one direction relative to the ratchet when the teeth engage in the ratchet element, so that, for example, a length of the bracing element or inelastic strap with a toothed rail construction can only be shortened in this state. To increase the length, the teeth must be disengaged from the ratchet element. This is possible, for example, by actuating a corresponding actuating element.

The knee element preferably comprises at least one swivel joint, the swivel axis of which, in the mounted state of the knee orthosis, coincides at least almost, but particularly preferably completely, with a swivel axis of the knee, wherein the knee element is preferably arranged on the swivel joint. Specifically, the arrangement of the knee element on the swivel joint and the adjustment element on the knee element ensures a special ease of handling, as it renders the adjustment element particularly easy to access. The individual elements themselves may be rigid or almost rigid components, for example made of a metal, a plastic or a fiber-reinforced plastic, and preferably have a cushion element which is arranged between the rigid component and the skin of the wearer.

The knee orthosis preferably features at least one, especially preferably two end stops, by way of which a swivelling of the swivel joint is limited in one, but preferably two directions. The end stops do not necessarily need to be designed to be symmetrical. It is definitely advantageous to allow a flexion of the knee joint to a greater extent than an extension. The reverse is of course also possible.

The knee orthosis preferably has a base body, in particular made of a textile, preferably a knitted fabric. The individual elements can be sewn, ironed, glued or otherwise applied to it. The at least one bracing element in particular preferably extends in at least one duct or tunnel that is arranged in the material of the base body or formed by applying, especially sewing, glueing or welding on, a further textile layer. If the at least one bracing element is situated inside this tunnel, it ensures that accidental contact, adjustment or even snagging is avoided.

Preferably, the knee orthosis comprises at least two upper leg contact elements and/or at least two lower leg contact elements.

The two end stops are preferably configured on a single end stop carrier, which is attached to a component of the swivel joint such that it can be detached. Conventionally, a swivel joint features at least two components that can be swivelled against each other about the swivel axis of the swivel joint in both swivel directions. The end stop carrier, which bears the two end stops, is arranged on one of the two components. A projection or end stop element is located on the other component that can be swivelled against the first component, wherein said projection or end stop element strikes one of the two end stops arranged on the end stop carrier when a certain swivel angle is reached, thereby preventing a swivel movement in the respective swivel direction.

Such a swivel joint, particularly with an end stop carrier described here, can be used and advantageously applied as a swivel joint not only in knee orthoses, but in almost all configurations of orthopedic devices. It therefore represents a separate, even isolated, invention from a knee orthosis. This isolated invention then relates to a swivel joint, in particular but not necessarily for a knee orthosis described here, wherein the ability to swivel is limited in one, preferably in two swivel directions, by two end stops configured on a single end stop carrier. Such a detached swivel joint may be designed with any one or any combination of the following features described for the swivel joint as part of the knee orthosis.

It is especially preferable if the end stop carrier is clamped, clipped or screwed onto the component, or plugged in a recess of the component. Irrespective of which specific type of fastening is selected, the end stop carrier is designed to be detachable. This means that it can be detached and in this state also removed from the respective component to which it is fastened. It is especially advantageous if it can be replaced in this state by another end stop carrier with different end stops. This is advantageous, for example, if the possible swivel range of an existing orthopedic device, particularly an existing knee orthosis, should be changed, especially increased, over time. In this case, it is not necessary to remove the swivel joint from the orthopedic device and replace it with a different joint with a different swivel range, i.e. in particular with differently positioned end stops. Rather, it is sufficient to detach the end stop carrier with the two end stops from the component to which it is fastened. In this state, it can be replaced with another end stop carrier.

The two end stops arranged on the end stop carrier can be two side walls of a projection, for example. The end stop element that is positioned on the other component of the swivel joint and strikes the respective end stop upon reaching the limit swivel angle comes into contact with the first end stop, i.e. the first side wall of the projection, upon reaching the one swivel angle in the first swivel direction. If the joint is swivelled in the opposite direction, the end stop element comes into contact with the second side wall of the projection on the end stop carrier when the second end stop angle is reached.

Due to the design as a detachable end stop carrier, the various swivel ranges defined by the respective end stop carrier and its two end stops are easily selectable and yet reproducible. It is only necessary to provide different end stop carriers for each desired swivel range.

In the following, an example of an embodiment of the present invention will be explained in more detail by way of the attached figures: They show:

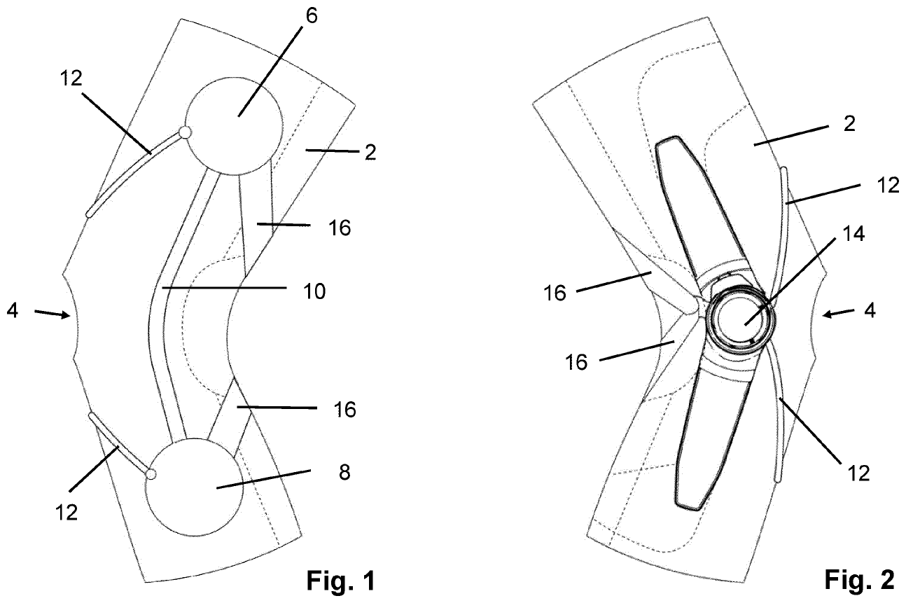

FIG. 1—the schematic lateral view of a knee orthosis in a medial top view,

FIG. 2—in a lateral top view, and

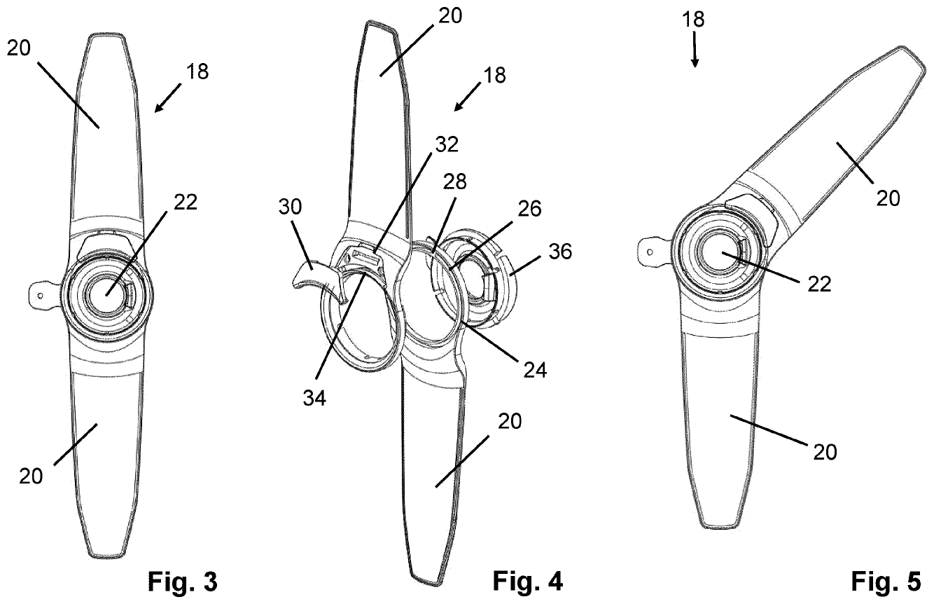

FIGS. 3-5—a swivel joint in a top view, an exploded view and a swivel position.

FIGS. 1 and 2 depict a knee orthosis according to an example of an embodiment of the present invention. It has a base body 2. The base body 2 can be made of an elastic, but at least flexible material, for example a woven or knitted fabric.

The base body 2 features a kneecap opening 4, so that the knee cap is not even covered in the mounted state.

FIG. 1 shows the medial side of the knee orthosis. An upper leg contact element 6 and a lower leg contact element 8 can be seen, each of which is arranged on the base body 2. A connecting element 10 extends between them, wherein said element can transmit compressive forces and is a rod spring, for example. One end of a bracing element 12 is shown on both the upper leg contact element 6 and the lower leg contact element 8. FIG. 2 shows a lateral view of the orthosis. A knee element 14 can be seen, towards which the two bracing elements 12 are guided. Of course, it may also be a single bracing element 12 that connects the upper leg contact element 8 via the knee element 14 to the lower leg contact element 8.

FIG. 1 also shows that an inelastic strap 16 leads away from both the upper leg contact element 6 and the lower leg contact element 8. In FIG. 2, it can be seen that the inelastic straps 16 are guided towards the knee element 14, where they are fastened. FIGS. 1 and 2 clearly demonstrate that the bracing element 12 extends exclusively in the ventral region of the knee orthosis, while the inelastic strap 16 is arranged in the dorsal region. The reverse embodiment is of course also possible.

FIG. 3 shows a top view of a swivel joint 18 which can be used specifically in a knee orthosis described here, but also in other orthopedic devices. It comprises two components 20 that are arranged in relation to each other such that they can be swivelled. A bore 22 is situated in the middle, the swivel axis passing through said bore.

FIG. 4 depicts the swivel joint 18 from FIG. 3 in an exploded view. A joint ring 24, which comprises an annular slit 26, is situated on the component 20 that is directed downwards. At one point, it is closed by an end stop element 28.

An end stop carrier 30 is located on the component 20 that is directed upwards, wherein said end stop carrier can be inserted into a purposefully provided recess 32 on the component 20. The recess 32 features an opening 34 through which the two end stops, not depicted in FIG. 4, can be guided. In the representation shown, they are on the rear side of the end stop carrier 30. It can be seen that, in the mounted state, the opening 34 comes to overlap with the annular slit 26 on the other component 20 so that the end stops, which are on the end stop carrier 30 and extend into the drawing plane through the opening 34, are arranged in the slit 26.

If the downward-facing component 20 is now swivelled relative to the upwardfacing component 20, the end stops of the end stop carrier 30 that protrude through the opening 34 are displaced relative to the annular slit 26 and thus also relative to the end stop element 28. In certain angular positions, which depend on the selected end stop carrier 30 with its fixed end stops, the end stop element 28 comes into contact with the respective end stop of the end stop carrier 30. A further swivelling in the respective swivel direction is then no longer possible, so that the swivel range of the swivel joint 18 is limited in this form.

The swivel joint 18 shown in FIG. 4 also has a fastening element 36 on which further components of the orthopedic device, such as a closing element, can be arranged.

FIG. 5 depicts the swivel joint 18 in a swivelled position relative to the embodiment shown in FIG. 4. The two components 20 are swivelled relative to each other about the swivel axis, which is situated in the bore 22.

### REFERENCE LIST

2 base body
4 kneecap opening
6 upper leg contact element
8 lower leg contact element
10 connecting element
12 bracing element
14 knee element
16 inelastic strap
18 swivel joint
20 component
22 bore
24 joint ring
26 slit
28 end stop element
30 end stop carrier
32 recess
34 opening
36 fastening element

1. A knee orthosis which comprises
a ventral section and a dorsal section,
an upper leg contact element,
a lower leg contact element and
a knee element,
at least one bracing element and at least one inelastic strap, characterized in that the upper leg contact element and the lower leg contact element are connected to the knee element by the at least one bracing element in the ventral region and by the at least on inelastic strap in the dorsal region, or vice-versa.

2. The knee orthosis according to claim 1, wherein the upper leg contact element and the lower leg contact element are connected by a connecting element, such as a rod, by way of which compressive forces can be transmitted.

3. The knee orthosis according to claim 2, wherein the connecting element is designed to be flexible in a direction perpendicular to the direction of extension running from the lower leg contact element to the upper leg contact element and/or has a corresponding joint.

4. The knee orthosis according to claim 1, wherein the knee orthosis comprises precisely one bracing element that extends from the upper leg contact element via the knee element to the lower leg contact element and/or precisely one inelastic strap that extends from the upper leg contact element via the knee element to the lower leg contact element.

5. The knee orthosis according to claim 1, wherein the at least one bracing element is designed to be adjustable in length.

6. The knee orthosis according to claim 5, wherein the knee orthosis features an adjustment element by way of which the length of the at least one bracing element can be adjusted, wherein the adjustment element is preferably situated on the knee element or is the knee element.

7. The knee orthosis according to claim 1, wherein the knee orthosis has at least two bracing elements, of which at least one extends from the upper leg contact element to the knee element and at least one extends from the knee element to the lower leg contact element and/or at least two inelastic straps, of which at least one extends from the upper leg contact element to the knee element and at least one extends from the knee element to the lower leg contact element.

8. The knee orthosis according to claim 1, wherein the at least one bracing element and/or the at least one inelastic strap comprises at least one strap, at least one wire, at least one cable, at least one toothed rail construction, and/or a cable pull.

9. The knee orthosis according to claim 1, wherein the knee element has at least one swivel joint whose swivel axis in the mounted state of the knee orthosis at least almost coincides with a swivel axis of the knee joint, wherein the knee element is preferably arranged on the swivel joint.

10. The knee orthosis according to claim 9, wherein the knee orthosis features at least one, but preferably two end stops, by way of which a swivelling of the swivel joint is limited in one, but preferably two directions, preferably in the sagittal plane.

11. The knee orthosis according to claim 1, wherein the knee orthosis comprises at least two upper leg contact elements and/or at least two lower leg contact elements.

12. The knee orthosis according to claim 10, characterized in that wherein the two end stops are configured on a single end stop carrier, which is fastened to a component of the swivel joint such that it can be detached.

13. The knee orthosis according to claim 12, wherein the end stop carrier is clamped, clipped or screwed onto the component, or plugged in a recess of the component.

14. A swivel joint, in particular for a knee orthosis according to claim 1, wherein the ability to swivel is limited in one, preferably in two swivel directions, by two end stops configured on a single end stop carrier.

15. A knee orthosis which comprises
a ventral section and a dorsal section;
an upper leg contact element;
a lower leg contact element;
a knee element; and
at least one bracing element and at least one inelastic strap;
wherein the upper leg contact element is connected to the lower leg contact element by a connecting element capable of transmitting a compressive force; and
wherein the upper leg contact element and the lower leg contact element are connected to the knee element by the at least one bracing element in the ventral region and by the at least on inelastic strap in the dorsal region, or vice-versa.

16. The knee orthosis according to claim 15, wherein the connecting element is designed to be flexible in a direction perpendicular to the direction of extension running from the lower leg contact element to the upper leg contact element and/or has a corresponding joint.

17. The knee orthosis according to claim 15, wherein the at least one bracing element extends from the upper leg contact element via the knee element to the lower leg contact element.

18. The knee orthosis according to claim 15, wherein the at least one bracing element is designed to be adjustable in length.

19. The knee orthosis according to claim 15, wherein the knee orthosis features an adjustment element by way of which the length of the at least one bracing element can be adjusted, wherein the adjustment element is preferably situated on the knee element or is the knee element.

20. A knee orthosis which comprises
a ventral section and a dorsal section;
an upper leg contact element;
a lower leg contact element;
a knee element;
at least one bracing element which is adjustable in length and at least one inelastic strap; and
an adjustment element for adjusting the at least one bracing element;
wherein the upper leg contact element is connected to the lower leg contact element by a connecting element capable of transmitting a compressive force; and
wherein the upper leg contact element and the lower leg contact element are connected to the knee element by the at least one bracing element in the ventral region and by the at least on inelastic strap in the dorsal region, or vice-versa.
